(19) United States
(12) Patent Application Publication
(10) Pub. No.: US 2024/0133857 A1
(43) Pub. Date: Apr. 25, 2024

(54) DEVICE AND METHOD FOR ON-SITE DETECTION OF SOIL ORGANIC MATTER, AND MICROFLUIDIC CHIP

(71) Applicant: ZHONGKE HEFEI INSTITUTES OF INTELLIGENT AGRICULTURE, Hefei (CN)

(72) Inventors: Rujing WANG, Hefei (CN); Jiangning CHEN, Hefei (CN); Xiangyu CHEN, Hefei (CN); Yongjia CHANG, Hefei (CN); Qinwen LU, Hefei (CN); Yang LIU, Hefei (CN); Yi LIU, Hefei (CN); Qiao CAO, Hefei (CN); Xiaoyu ZHANG, Hefei (CN)

(21) Appl. No.: 18/396,388

(22) Filed: Dec. 26, 2023

Related U.S. Application Data
(63) Continuation of application No. PCT/CN2022/128072, filed on Oct. 27, 2022.

(30) Foreign Application Priority Data
Jul. 4, 2022 (CN) 202210783531.7
Jul. 4, 2022 (CN) 202210787357.3

(51) Int. Cl. G01N 33/24; B01L 3/00; G01N 21/31

(52) U.S. Cl. CPC G01N 33/24; B01L 3/502707; B01L 3/502753; G01N 21/31; B01L 2200/027; B01L 2200/12; B01L 2300/0681; B01L 2300/0803; B01L 2300/1805; B01L 2400/0409

(57) ABSTRACT

A device for on-site detection of soil organic matter, including a pre-processing module, a centrifugal system, a microfluidic chip, and a photoelectric detection module. The pre-processing module is configured to process a soil sample into a soil solution. The centrifugal system is configured to generate a centrifugal force. The microfluidic chip is configured to allow mixing of the soil solution and an extraction solvent for extraction under the centrifugal force to obtain an extract. The photoelectric detection module is configured to detect the extract to determine organic matter content in the soil solution. An on-site detection method and a microfluidic chip are also provided. The microfluidic chip includes a channel layer, a cover layer arranged above the channel layer, and a base plate layer arranged below the channel layer.

### CROSS-REFERENCE TO RELATED APPLICATIONS

This application is a continuation of International Patent Application No. PCT/CN2022/128072, filed on Oct. 27, 2022, which claims the benefit of priority from Chinese Patent Application Nos. 202210787357.3 and 202210783531.7, both filed on Jul. 5, 2022. The content of the aforementioned application, including any intervening amendments thereto, is incorporated herein by reference in its entirety.

### TECHNICAL FIELD

This application relates to detection of soil organic matter, and more particularly to a device and method for on-site detection of soil organic matter, and a microfluidic chip.

### BACKGROUND

Scientific management of fertilization and improvement of food production are two major challenges in the modern agricultural production. Organic matter is an important component of soil, and is an important indicator of soil fertility, providing crops with essential nutrients. The soil fertility can be estimated by detecting the content of soil organic matter. The soil fertility can be improved by supplementing soil nutrients through precise fertilization, so as to improve the crop yield and quality.

The soil organic matter content is generally detected through traditional chemical methods, such as potassium dichromate volumetric method, dry combustion method, and loss-on-ignition method. These traditional methods require complex and cumbersome manual operations for soil samples, and thus are inefficient, costly, and time-consuming. Besides, the involved detection equipment is expensive and bulky, and requires regular maintenance. Therefore, such traditional methods are not suitable for rapid on-site detection and analysis. With the development of science and technology, various advanced techniques, such as near-infrared spectroscopy, remote sensing and hyperspectral technique, have been widely used in the rapid estimation of soil nutrients in recent years. These methods involve the construction of a model for estimating the soil organic matter content, and for various soil samples, multiple estimation models are required, failing to achieve the rapid and accurate model construction.

Microfluidics, as a new analytical platform, has the advantages of miniaturization, automation, integration, simplification, and rapid detection, and thus have been widely applied in the practical detection. However, the centrifugal microfluidic analysis technology, in which several samples in the form of micro fluid are simultaneously detected in a disc-shaped chip under the drive of centrifugal force, has still not be reported in the on-site rapid detection of soil organic matter.

Therefore, there is an urgent need for a low-cost, rapid, and reliable method for on-site detection of soil organic matter and related chips.

### SUMMARY

An object of the present disclosure is to provide a device and method for on-site detection of soil organic matter, and a microfluidic chip, so as to overcome the deficiencies in the prior art, and achieve the on-site detection of soil organic matter with less time consumption, higher detection efficiency, and simplified operation.

The technical solutions of the present disclosure are described below.

In a first aspect, this application provides device for on-site detection of soil organic matter, comprising:
a pre-processing module;
a centrifugal system;
a microfluidic chip; and
a photoelectric detection module;
wherein the pre-processing module is configured to process a soil sample into a soil sample solution;
the centrifugal system is configured to generate a centrifugal force to drive fluids in the microfluidic chip to move towards a periphery of the microfluidic chip;
the microfluidic chip is configured for flowing and mixing of the soil sample solution and an extraction solvent within the microfluidic chip for extraction under an action of the centrifugal force generated by the centrifugal system to obtain an extract; and
the photoelectric detection module is configured to detect the extract to determine organic matter content in the soil sample solution.

In some embodiments, the device further comprises:
a microprocessor module;
a heating plate;
a temperature control module;
a drive module;
a display;
a power supply module; and
a communication module;
wherein the heating plate is arranged below the microfluidic chip, and is configured to heat the microfluidic chip; the temperature control module is configured to control a heating temperature of the heating plate; the drive module is configured to drive the centrifugal system to work; the power supply module is configured for supplying power to the microprocessor module; the communication module is configured for realizing communication of the microprocessor module with other modules; the display is configured for displaying detection results of the photoelectric detection module; and the microprocessor module is configured to receive and analyze the detection results of the photoelectric detection module and display analysis results on the display, and control the drive module and the temperature control module.

In some embodiments, the pre-processing module is configured to process a solvent solution to obtain the extraction solvent.

In some embodiments, the centrifugal system is a centrifugal detector.

In some embodiments, the microfluidic chip comprises a channel layer and a cover layer arranged above the channel layer;
wherein the channel layer comprises a channel layer main body and a plurality of channel branches arranged on the channel layer main body; each of the plurality of channel branches comprises a first solvent injection port, a sample inlet, an extraction well, a microchannel, a filtration well, a detection well, a waste collection well and a first air hole; an inlet of the extraction well is connected to the first solvent injection port and the sample inlet; an outlet of the extraction well is connected to an inlet of the microchannel; an outlet of the microchannel is connected to an inlet of the filtration well; an outlet of the filtration well is connected to an inlet of the detection well; an outlet of the detection well is connected to an inlet of the waste collection well; the waste collection well is communicated with the first air hole; the extraction well is provided with a plurality of heating columns; the filtration well is provided with a microarray and a plurality of microbeads; the plurality of microbeads are located above the microarray; and a filter pad is provided at the outlet of the filtration well.

In some embodiments, the cover layer is provided with a first mounting hole, a plurality of sample injection holes, a plurality of second solvent injection ports and a plurality of second air holes; a middle of the channel layer main body is provided with a second mounting hole corresponding to the first mounting hole; the plurality of sample injection holes, the plurality of second solvent injection ports, the plurality of second air holes and the plurality of channel branches are the same in number, and the plurality of channel branches are in one-to-one correspondence with the plurality of sample injection holes, the plurality of second solvent injection ports and the plurality of second air holes; the plurality of sample injection holes are in one-to-one correspondence with sample inlets of the plurality of channel branches; the plurality of second air holes are in one-to-one correspondence with first air holes of the plurality of channel branches; and the plurality of second solvent injection ports are in one-to-one correspondence with first solvent injection ports of the plurality of channel branches.

In some embodiments, the cover layer is provided with an observation window; and the observation window comprises a penetration hole arranged on the cover layer and an optically-permeable film mounted in the penetration hole.

In some embodiments, a base plate layer is provided below the channel layer; and a middle of the base plate layer is provided with a third mounting hole.

In some embodiments, the filter pad is configured as at least one layer; and the filter pad is a metal filter screen, a non-metallic filter cloth, a filter membrane, or a combination thereof.

In a second aspect, this application provides a detection method using the aforementioned on-site detection device, comprising:
(S1) processing, by the pre-processing module, a soil sample to obtain a soil sample solution;
(S2) injecting the soil sample solution and an extraction solvent into the microfluidic chip;
(S3) heating the microfluidic chip using a heating plate;
(S4) starting the centrifugal system to generate a centrifugal force to drive the soil sample solution and the extraction solvent in the microfluidic chip to flow along a channel branch for mixing and extraction, so as to obtain an extract; and
(S5) detecting, by the photoelectric detection module, the extract to determine the organic matter content in the soil sample solution.

In a third aspect, this application provides a centrifugal microfluidic chip, comprising:
a channel layer;
a cover layer arranged above the channel layer; and
a base plate layer arranged below the channel layer;
wherein the channel layer comprises a channel layer main body and a plurality of channel branches arranged on the channel layer main body; each of the plurality of channel branches comprises a sample inlet, an extraction well, a microchannel, a filtration well, a detection well, and a waste collection well; an inlet of the extraction well is connected to the sample inlet; an outlet of the extraction well is connected to an inlet of the microchannel; an outlet of the microchannel is connected to an inlet of the filtration well; an outlet of the filtration well is connected to an inlet of the detection well; and an outlet of the detection well is connected to an inlet of the waste collection well.

In some embodiments, the extraction well is provided with a plurality of heating columns.

In some embodiments, the filtration well is provided with a microarray and a plurality of microbeads; the plurality of microbeads are located above the microarray; and a filter pad is provided at the outlet of the filtration well.

In some embodiments, the cover layer is provided with a first mounting hole, a plurality of sample injection holes, a plurality of first solvent injection ports, and a plurality of first air holes.

In some embodiments, each of the plurality of channel branches further comprises a second air hole connected to the waste collection well and a second solvent injection port connected to the inlet of the extraction well; a middle of the channel layer main body is provided with a second mounting hole corresponding to the first mounting hole; the plurality of sample injection holes, the plurality of first solvent injection ports, the plurality of first air holes, and the plurality of channel branches are the same in number, and the plurality of channel branches are in one-to-one correspondence with the plurality of sample injection holes, the plurality of first solvent injection ports and the plurality of first air holes; the plurality of sample injection holes are in one-to-one correspondence with sample inlets of the plurality of channel branches; the plurality of first air holes are in one-to-one correspondence with second air holes of the plurality of channel branches; and the plurality of first solvent injection ports are in one-to-one correspondence with second solvent injection ports of the plurality of channel branches.

In some embodiments, the cover layer is provided with a observation window; and the observation window comprises a penetration hole arranged on the cover layer and an optically-permeable film mounted in the penetration hole.

In some embodiments, a middle of the base plate layer is provided with a third mounting hole.

In some embodiments, the channel layer comprises a first channel layer and a second channel layer arranged in sequence; an upper portion of each of the plurality of channel branches is located in the first channel layer, and a lower portion of each of the plurality of channel branches is located in the second channel layer; and the upper portion is through in the first channel layer and is communicated with the lower portion in the second channel layer.

In some embodiments, the filter pad is configured as at least one layer; and the filter pad is a metal filter screen, a non-metal filter cloth, a filter membrane, or a combination thereof.

In a fourth aspect, this application provides a method for preparing the centrifugal microfluidic chip, comprising:
(a) drawing patterns of microstructures required on the cover layer, the channel layer and the base plate layer by using a computer software;
(b) forming the microstructures on the cover layer, the channel layer and the base plate layer by microfabrication; and
(c) subjecting the cover layer, the channel layer and the base plate layer to alignment, bonding, and pressurized sealing to fabricate the centrifugal microfluidic chip.

Compared to the prior art, this application has the following beneficial effects.
(1) This application adopts a combination of a microfluidic chip and an automated portable instrument to realize the full integration and automation of chemical reactions and detection of soil organic matter. Moreover, it is easy to operate and miniaturize, and is suitable for on-site and rapid organic matter detection of batches of soil samples.
(2) With respect to the detection of soil organic matter, this application integrates extraction, reaction, separation, and color development processes inside the microfluidic chip, reducing the consumption of sample and solvent, lowering the costs and improving the detection efficiency. With the help of a centrifugal microfluidic chip, multiple samples can be analyzed in parallel at the same time, suitable for the batch analysis. Different channel branches can be used for the detection and analysis of different samples.
(3) The extraction process of soil organic matter in an alkaline solution requires heating. In this application, a heating plate is placed below the microfluidic chip, and the metallic heating column is provided in the extraction well, so that the extraction system in the extraction well can be heated quickly to the specified temperature, thereby improving the extraction efficiency and detection accuracy.
(4) Regarding the structural design of the microfluidic chip, a filtration well is provided between the extraction well and the detection well, and a microarray and several microbeads with different sizes are arranged in the filtration well. Besides, a filter pad is arranged at the outlet of the filtration well. Through the mutual collaboration of the microbeads, the microarray, and the filter pad, the fine particles in the to-be-tested liquid can be effectively filtered out, avoiding the interference of impurities to the subsequent detection, and effectively improving the detection accuracy and reliability.

### BRIEF DESCRIPTION OF THE DRAWINGS

FIG. 1 is a block diagram of an on-site detection device of soil organic matter according to an embodiment of the present disclosure;

FIG. 2 is an exploded view of a microfluidic chip according to an embodiment of the present disclosure;

FIG. 3 is an exploded view of a microfluidic chip according to another embodiment of the present disclosure;

FIG. 4 is a schematic diagram of a cover layer according to an embodiment of the present disclosure;

FIG. 5 is a schematic diagram of a channel layer (channel layer I) according to an embodiment of the present disclosure;

FIG. 6 is a schematic diagram of a channel layer (channel layer II) according to another embodiment of the present disclosure;

FIG. 7 is a top view of a channel branch according to an embodiment of the present disclosure; and

FIG. 8 is a perspective view of the channel branch according to an embodiment of the present disclosure.

In the drawings, 1, microprocessor module; 2, centrifugal system; 3, pre-processing module; 4, photoelectric detection module; 5, temperature control module; 6, display; 7, power supply module; 8, communication module; 9, microfluidic chip; 10, heating plate; 11, drive module; 901, cover layer; 902, channel layer; 903, channel branch; 904; first air hole; 905, sample injection hole; 906, first solvent injection port; 907, sample inlet; 908, second solvent injection port; 909, heating column; 910, extraction well; 911, microchannel; 912, microarray; 913, microbead; 914, filter pad; 915, filtration well; 916, detection well; 917, waste collection well; and 918, second air hole.

### DETAILED DESCRIPTION OF EMBODIMENTS

The present disclosure will be further described below with reference to the embodiments and accompanying drawings.

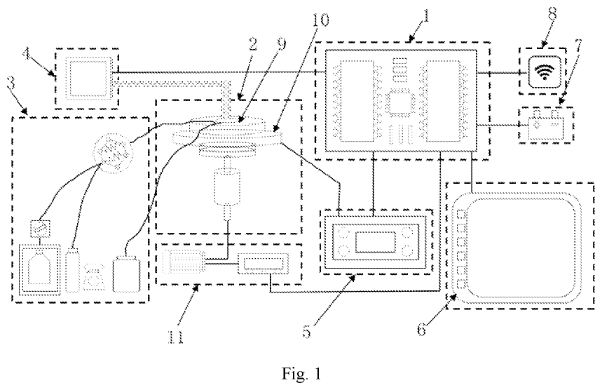

As shown in FIG. 1, this application provides an on-site detection device for soil organic matter, including: a pre-processing module 3, a centrifugal system 2, a microfluidic chip 9, a photoelectric detection module 4, a microprocessor module 1, a heating plate 10, a temperature control module 5, a drive module 11, a display 6, a power supply module 7, and a communication module 8. The on-site detection device described herein integrates the functions of soil pre-treatment, injection, the reaction of soil sample solution with extraction solvent, detection, and analysis of detection results, thereby realizing the manipulation of the microfluidic chip and the on-site rapid detection and analysis of soil organic matter. Based on the principle of colorimetric detection of organic matter, the detection device provided herein realizes automatic, rapid, and accurate detection of soil organic matter.

The pre-processing module 3 is configured to process a soil sample into a soil sample solution and process an extraction solvent into an extraction solvent solution. The pre-processing module 3 includes a through-valve, a quantification ring, a soil sample processing device, a solvent processing device, a scale, and a waste liquid processing device. The scale is configured for weighing. The through-valve is configured for feeding a sample. The quantification ring is configured for quantifying. The soil sample processing device is configured for converting a soil sample into a soil sample solution. The solvent processing device is configured for preparing a solvent solution. The waste liquid processing device is configured for treating the waste liquid.

The centrifugal system 2 is configured to generate a centrifugal force. The centrifugal system is a centrifugal detector. The drive module is configured to drive the centrifugal system to work. The centrifugal system and drive module are configured to achieve precise manipulation and transfer of the liquid on the microfluidic chip by using a rotating tray and a centrifugal microfluidic solvent tray, driven by centrifugal force.

The microfluidic chip 9 is configured for the flowing and mixing of the soil sample solution and the extraction solvent within the microfluidic chip for extraction under the action of the centrifugal force generated by the centrifugal system.

The photoelectric detection module 4 is configured to detect the extract to determine the organic matter content in the soil sample solution. The photoelectric detection module 4 includes a light source and a photoelectric sensor. By measuring the absorbance of the detection zone (color development zone) on the microfluidic chip 9, the photoelectric detection module 4 inputs the detected signals into the microprocessor module 1 for data processing to obtain test results. The soil organic matter content is determined according to the test results, and the precise test results are displayed on the display 6, or stored and printed through the communication module 8.

The heating plate 10 is arranged below the microfluidic chip 9 and is configured to heat the microfluidic chip 9. The temperature control module 5 is configured to control the heating temperature of the heating plate 10. The temperature control module 5 is configured to control the overall temperature of the microfluidic chip 9 by arranging the heating plate 10 and the temperature sensor at the bottom of the microfluidic chip 9.

The power supply module 7 is configured for supplying power to the microprocessor module 1.

The communication module 8 is configured for realizing communication of the microprocessor module 1 with other modules.

The display 6 is configured for displaying detection results of the photoelectric detection module.

The microprocessor module 1 is configured to receive and analyze the detection results of the photoelectric detection module 4 and display analysis results on the display 6, and control the drive module 11 and the temperature control module 5. The microprocessor module 1, the communication module 8 and the display 6 together achieve the control on the whole device, data acquisition, detection analysis, result display and printing, and transmission processing.

Through microfluidics technology, basic operation units, such as sample preparation, reaction, separation, and detection, can be made into micron and nanometer-scale components, and these components can be integrated into a tiny chip, thereby realizing the whole process of analysis and testing. As the microfluidic chip requires a small amount of sample and solvent and has high detection efficiency, it has been widely used in testing-related fields as a new analytical platform with the advantages of miniaturization, automation, integration, convenience, and rapidity. In this application, the microfluidic chip technology is applied to soil organic matter detection to develop a fully automated, fast, and simple detection platform and analysis method, which significantly saves time for sample processing and minimizes the cost of solvents and instruments. Moreover, it can also achieve intelligent rapid detection in the form of “sample in, result out” while ensuring accurate detection for the whole process. Therefore, it is significant to perform rapid and accurate analysis and determination on the soil organic matter and solve the problem of on-site rapid measurement of soil organic matter.

The centrifugal microfluidic chip includes a channel layer, a cover layer arranged above the channel layer, and a bottom layer arranged below the channel layer.

The channel layer includes a channel layer main body and multiple channel branches arranged on the channel layer main body. Each channel branch includes a sample inlet, an extraction well, a microchannel, a filtration well, a detection well, and a waste collection well. An inlet of the extraction well is connected to the sample inlet, and an outlet of the extraction well is connected to an inlet of the microchannel. An outlet of the microchannel is connected to an inlet of the filtration well. An outlet of the filtration well is connected to an inlet of the detection well, and an outlet of the detection well is connected to an inlet of the waste collection well.

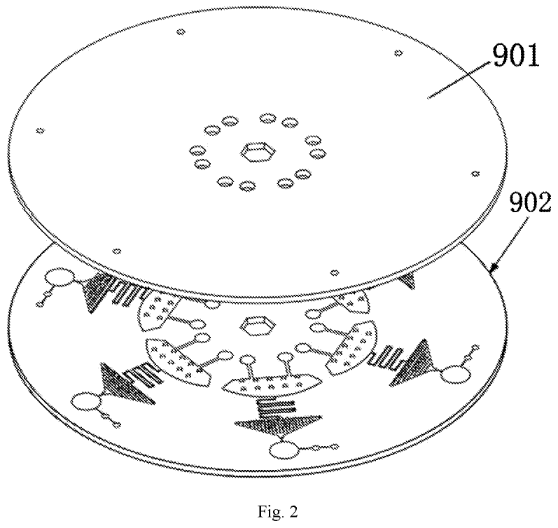

In an embodiment, a microfluidic chip 9 as shown in FIG. 2 is formed by a plurality of circular sheets bonded together, and includes a channel layer 902 and a cover layer 901 arranged above the channel layer 902.

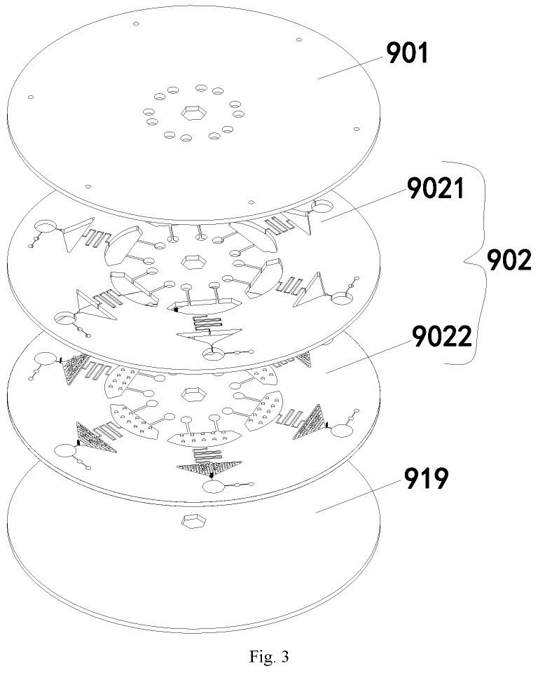

In another embodiment, a microfluidic chip 9 as shown in FIG. 3 further includes a bottom plate 919 arranged below the channel layer 902. The bottom plate 919 is configured to reinforce the channel layer. To facilitate processing, the channel layer 902 can be designed as a two-layer structure, with the upper layer being the channel layer I 9021 and the lower layer being the channel layer II 9022.

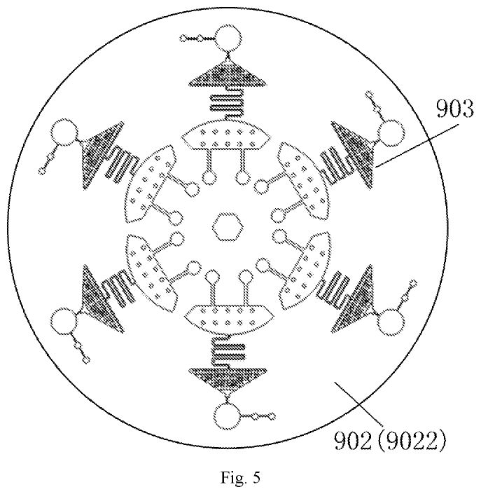

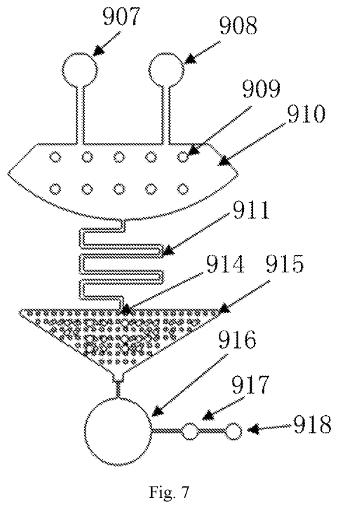

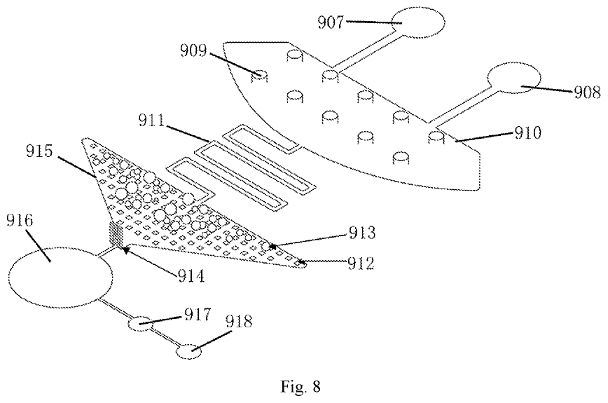

As shown in FIGS. 5-8, the channel layer 902 includes a channel layer main body and a plurality of channel branches 903 provided on the channel layer main body. Under the control of centrifugal force, the soil sample solution and the extraction solvent can be precisely manipulated and transferred inside the microfluidic chip 9 to achieve mixing, reaction, separation and color development of the soil sample solution and the extraction solvent.

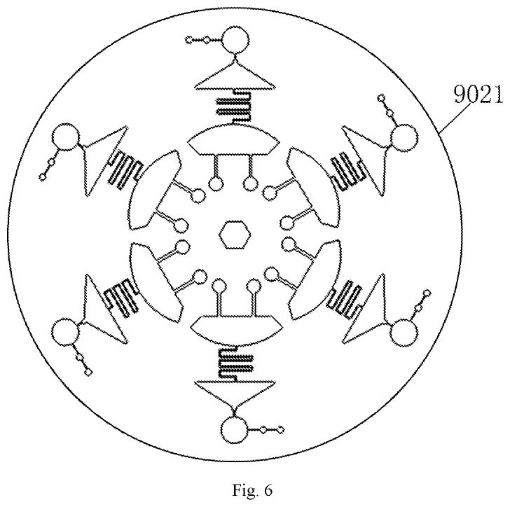

FIG. 5 is a schematic diagram of a channel layer 902 (channel layer II 9022 of the second embodiment) of the first embodiment of the present disclosure. FIG. 6 is a schematic diagram of a channel layer I 9021 of the second embodiment of the present disclosure.

As shown in FIGS. 2 and 4-8, in the first embodiment, the channel layer 902 has only one layer structure. In contrast, in the second embodiment, the channel layer 902 can be designed into a two-layer structure, namely, the upper channel layer I 9021 and the lower channel layer II 9022.

The channel branch 903 has an upper portion located in channel layer I 9021 and a lower portion located in channel layer II 9022. The upper portion of the channel branch 903 is through in the channel layer I, and is connected to the channel branch in the channel layer II. The channel branch 903 includes a second solvent injection port 908, a sample inlet 907, an extraction well 910, a microchannel 911, a filtration well 915, a detection well 916, a waste collection well 917, and a second air hole 918. An inlet of the extraction well 910 is connected to the second solvent injection port 908 and the sample inlet 907, respectively. The outlet of extraction well 910 is connected to the inlet of microchannel 911. The outlet of the microchannel 911 is connected to the inlet of the filtration well 915. The outlet of the filtration well 915 is connected to an inlet of the detection well 916. The outlet of the detection well 916 is connected to the inlet of the waste collection well 917, and the waste collection well 917 is connected to the second air hole 918. Under the control of centrifugal force, the soil sample solution and the extraction solvent can be precisely manipulated and transferred inside the microfluidic chip to achieve mixing, reaction, separation and color development of the soil sample solution and the extraction solvent.

The extraction well 910 is provided with a plurality of heating columns 909. The heating columns 909 are configured to heat the mixture of sample and extraction solvent in the extraction well 910 such that the sample and solvent can react more fully in the extraction well under heated conditions. The heating plate is configured to heat the entire microfluidic chip and the liquid therein to ensure that the mixture can react fully throughout the process.

The filtration well 915 is provided with a microarray 912 and a plurality of microbeads 913 varied in sizes. The microbeads 913 are located above the microarray 912, and the microarray 912 can be directly processed into a square microcolumn array in the filtration well 915. The filtration well 915 can be provided with the microarray, the plurality of microbeads, or a combination thereof. In addition, a filter pad 914 is provided at the outlet of the filtration well 915, and the filter pad 914 is a metal filter screen, a non-metal filter cloth, a filter membrane, or a combination thereof, which can be configured as a layer or multiple layers, depending on the situation. The number of the microsphere 913 is more than ore, and the plurality of microbeads 913 are randomly arranged in the filtration well 915 and are varied in size. The microbeads 913 are organic polymers, polystyrene (PS) microbeads, or silica microbeads synthesized in situ. The microarray 912, the microbeads 913 and the filter pad 914 are collaborative to effectively filter out fine particles from the liquid to be measured. The filtration well 915 can effectively remove particles with different sizes from the extract through the filtration structures of the microarray 912, microbeads 913 and the filter pad 914 for secondary filtration of the extract to be tested, thus improving the accuracy of the test results.

When being tested, the sample solution is introduced through the sample injection hole 905, and the extraction solvent is introduced through the first solvent injection port 906 and the second solvent injection port 908. Then the sample solution and the extraction solvent flow through the extraction well 910, the filtration well 915 and the detection well 916 along the microchannel 911 to complete the color development reaction of the solution. The solvent can be liquid or solid. If the solvent is liquid, it can be introduced under pressure in the pre-processing module 3 or encapsulated inside the microfluidic chip 9 in the form of a liquid capsule. If the solvent is solid, it can be powder or a block solvent sealed inside the microfluidic chip 9.

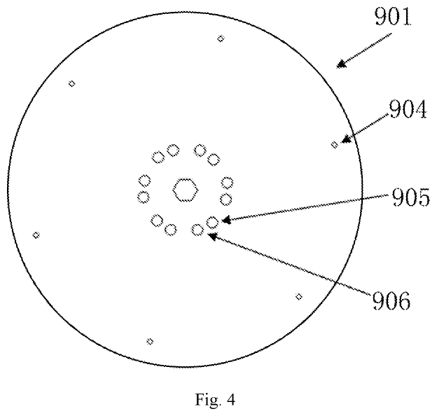

As shown in FIG. 4, the cover layer 901 is provided with a mounting hole I, several sample injection holes 905, several first solvent injection ports 906 and several first air holes 904. The channel layer main body is provided with a mounting hole II in the middle corresponding to the mounting hole I. A mounting hole III is provided in the middle of the base plate layer. The mounting hole I, the mounting hole II, and the mounting hole III are provided corresponding to each other, and are used for mounting the microfluidic chip on the centrifugal detector.

The sample injection hole 905, the first solvent injection port 906, the first air hole 904, and the channel branch 903 are provided in equal numbers and in correspondence. The sample injection hole 905 and the sample inlet 907 are arranged in correspondence and are communicated with each other. The soil sample solution is added from the sample injection hole 905, and then flows into the sample inlet 907 along the sample injection hole 905. The first air hole 904 is provided in correspondence with the second air hole 918 to allow the interior of channel branch 903 to communicate with the outside atmosphere to maintain the pressure balance. The first solvent injection port 906 and the second solvent injection port 908 are corresponding and communicated with each other. The solvent is added from the first solvent injection port 906, and flows to the second solvent injection port 908 along the first solvent injection port 906.

Furthermore, the cover layer 901 is provided with an observation window. The observation window includes a penetration hole arranged on the cover layer and an optically-permeable film mounted in the penetration hole. The photoelectric detection module 4 emits detection light through the observation window to optically detect the reaction results in the detection well.

The following is a method for preparing and using the aforementioned centrifugal microfluidic chip, which includes the following steps.
(1) Patterns of microstructures required on each layer of the centrifugal microfluidic chip are designed and drawn with a computer-assisted design software. The microstructures include air holes, mounting holes, sample inlets, microchannels, extraction wells, filtration wells, detection wells, and waste collection wells.
(2) Desired microstructures are formed on the surface of each of the cover layer, the channel layer, and the base plate layer of the microfluidic chip by microfabrication.
(3) An extraction solvent is placed in the reaction well or is injected through the solvent injection port while performing the test.
(4) Individual layers of the centrifugal microfluidic chip, such as the cover layer, the channel layer, and the base plate layer, are aligned, bonded, and sealed under pressure by using a bonding technique to fabricate the centrifugal microfluidic chip.
(5) After the centrifugal microfluidic chip is sealed, a to-be-tested sample solution is added from the sample injection hole.
(6) The centrifugal microfluidic chip is mounted on the centrifuge through the mounting hole, and the centrifuge is started. The to-be-tested sample solution and the extractant are mixed, reacted and separated in the extraction well under the action of centrifugal force.
(7) The centrifugal speed of the centrifuge is changed such that the extractant is transferred from the extraction well to the filtration well through a microvalve to undergo solid-liquid separation, where the microvalve is installed between the extraction well and the filtration well.
(8) The centrifugal speed of the centrifuge is changed again such that the clarified liquid after filtration is transferred into the detection well through a microvalve, where the microvalve is installed between the filtration well and the detection well.
(9) After the sample is processed, the extracted solution is detected by a photoelectric detector to obtain the content of organic matter.

The working process of the microfluidic chip provided in the present disclosure is described as follows.

When the microfluidic chip 9 is used for soil organic matter detection, the soil sample solution is injected into the microfluidic chip 9 through the sample injection hole 905, and then flows to the sample inlet 907 from the sample injection hole 905. The solvent is injected from the first solvent injection port 906 or is pre-built in the first solvent injection port 906 and the second solvent injection port 908.

This centrifugal microfluidic chip is configured for detecting soil organic matter, which is a disc-shaped chip consisting of multiple layers of chips. Driven by the centrifugal force generated by rotation, the mixing, reaction, separation and color development process of the to-be-tested sample and the reaction reagent are realized. Finally, the organic matter content in the soil sample is quantitatively detected by UV-visible spectrophotometer. This centrifugal microfluidic chip for soil organic matter detection requires a small amount of sample and reagents and can process and test multiple samples in parallel, which is fast and convenient.

Under the action of centrifugal force generated by the centrifugal system, the soil sample solution in the sample inlet 907 flows into the extraction well 910 to mix with the extraction solvent. The heating column 909 in the extraction well 910 heats the mixture of the soil sample solution and the extraction solvent in the extraction well 910 to accelerate the extracting rate. The soil sample solution and the extraction solvent undergo an initial extraction in the extraction well, and continue to move forward under the centrifugal force of the centrifugal system 2 to flow in the spiral or round-trip bent-shaped microchannels 911 for extraction. By designing the microchannels 911 in a spiral or round-trip bend shape, the soil sample solution can be fully extracted with the solvent.

With centrifugal force as the driving force, the microfluid can be precisely manipulated and transferred to achieve the mixing, reaction, separation and color development of the sample and the reaction reagent on the chip. The content of organic matter in the sample is qualitatively or quantitatively detected by the photoelectric detector, which can realize the on-site fast detection of soil organic matter with short detection cycle, high detection efficiency, simple and convenient operations.

When the mixture of soil sample solution and solvent flows to the filtration well, the mixture moves forward along the horizontal direction to filter the excess particulate matter through microbeads 913 and microarray 912 while moving from top to bottom, during which the mixture passes through the microbeads 913 to filter the excess particulate matter therein and then passes through the microarray 912 to filter the particulate matter. When the mixture flows to the filter pad 914 at the outlet of the filtration well 915, the filter pad 914 filters the mixture once more to filter out the excess particulate matter therein. Through the multiple filtrations of the microarray 912, the microbeads 913 and the filter pad 914, the excess particulate matter in the mixture can be filtered out as much as possible to ensure the accuracy of the test results.

After multiple filtration processes, the mixture flows into the detection well 916, and the extracted liquid is tested by the photoelectric detection module 4 to determine the organic matter content in the soil. After the test is completed, the liquid flows to the waste collection well. During the flowing of the mixture, the extraction keeps going between the soil sample solution and the solvent.

This application also provides a detection method by using the above-mentioned detection device, which includes the following steps.
(1) A soil sample is processed by the pre-processing module 3 to obtain a soil sample solution.
(2) The soil sample solution and an extraction solvent are injected into the microfluidic chip 9.
(3) The microfluidic chip 9 is heated with the heating plate 10.
(4) The centrifugal system 2 works to generate a centrifugal force. The soil sample solution and the extraction solvent in the microfluidic chip 9 is driven by the centrifugal force to flow along the channel branch 903 for mixing and extraction to obtain an extract.

The centrifugal system is started such that the to-be-tested sample solution and the extraction solvent are mixed, reacted and separated in the extraction well under the centrifugal force. The centrifugal speed of the centrifugal system is changed such that the extract is transferred from the extraction well to the filtration well through a microvalve for solid-liquid separation, where the microvalve is installed between the extraction well and the filtration well. The centrifugal speed of the centrifugal system is changed again such that the clarified liquid after filtration is transferred into the detection well through a microvalve, where the microvalve is installed between the filtration well and the detection well.
(5) The extract is detected by the photoelectric detection module 4 to determine the organic matter content in the soil sample solution.

The beneficial effects of the present disclosure are described below.
(1) This application adopts a combination of a microfluidic chip and an automated portable instrument to realize the full integration and automation of chemical reactions and detection of soil organic matter. Moreover, it is easy to operate and miniaturize, and is suitable for on-site and rapid organic matter detection of batches of soil samples.
(2) With respect to the detection of soil organic matter, this application integrates extraction, reaction, separation, and color development processes inside the microfluidic chip, reducing the consumption of sample and solvent, lowering the costs and improving the detection efficiency. With the help of a centrifugal microfluidic chip, multiple samples can be analyzed in parallel at the same time, suitable for the batch analysis. Different channel branches can be used for the detection and analysis of different samples.
(3) The extraction process of soil organic matter in an alkaline solution requires heating. In this application, a heating plate is placed below the microfluidic chip, and the metallic heating column is provided in the extraction well, so that the extraction system in the extraction well can be heated quickly to the specified temperature, thereby improving the extraction efficiency and detection accuracy.
(4) This application adopts a centrifugal microfluidic to complete the mixing, extraction, and detection process of the to-be-tested sample and the extraction solvent. with a centrifugal force as the driving force. Compared with the prior art, the microfluidic chip and method provided herein can simultaneously process and detect multiple samples, which requires a few samples and reagents, achieves full integration and automation of chemical reactions and detection, and have advantages of economy, rapidity, portability, and efficiency, providing a new analytical technology platform for detecting soil organic matter and meeting the demand for on-site rapid screening of soil organic matter.
(5) In this application, the extraction, reaction, separation, and color development processes of soil organic matter are carried out inside the microfluidic chip, which requires a small amount of sample and solvent and has low costs and high detection efficiency. With the centrifugal microfluidic chip, multiple samples can be analyzed in parallel at the same time, which is especially suitable for screening large-volume samples. Different channel branches can be used for the detection and analysis of different samples.
(6) The extraction process of soil organic matter in alkaline solution requires heating. In this application, a heating plate is placed below the microfluidic chip in which the heating column in the extraction well is made of metal, so that the solution in the extraction well can be heated quickly to the specified temperature, thereby improving the extraction effects and detection accuracy.
(7) Regarding the structural design of the microfluidic chip, a filtration well is provided between the extraction well and the detection well, and a microarray and several microbeads with different sizes are arranged in the filtration well. Besides, a filter pad is arranged at the outlet of the filtration well. Through the mutual collaboration of the microbeads, the microarray, and the filter pad, the fine particles in the to-be-tested liquid can be effectively filtered out, avoiding the interference of impurities to the subsequent detection, and effectively improving the detection accuracy and reliability.

Described above are merely preferred embodiments of the present disclosure, which are not intended to limit the scope of the present disclosure. Without departing from the spirit of the present disclosure, all variations and improvements made to the technical solutions of the present disclosure by one of ordinary skill in the art shall fall within the scope of the disclosure defined by the appended claims.

What is claimed is:

1. A device for on-site detection of soil organic matter, comprising:
a pre-processing module;
a centrifugal system;
a microfluidic chip; and
a photoelectric detection module;
wherein the pre-processing module is configured to process a soil sample into a soil sample solution;
the centrifugal system is configured to generate a centrifugal force to drive fluids in the microfluidic chip to move towards a periphery of the microfluidic chip;
the microfluidic chip is configured for flowing and mixing of the soil sample solution and an extraction solvent within the microfluidic chip for extraction under an action of the centrifugal force generated by the centrifugal system to obtain an extract; and
the photoelectric detection module is configured to detect the extract to determine organic matter content in the soil sample solution.

2. The device of claim 1, further comprising:
a microprocessor module;
a heating plate;
a temperature control module;
a drive module;
a display;
a power supply module; and
a communication module;
wherein the heating plate is arranged below the microfluidic chip, and is configured to heat the microfluidic chip; the temperature control module is configured to control a heating temperature of the heating plate; the drive module is configured to drive the centrifugal system to work; the power supply module is configured for supplying power to the microprocessor module; the communication module is configured for realizing communication of the microprocessor module with other modules; the display is configured for displaying detection results of the photoelectric detection module; and the microprocessor module is configured to receive and analyze the detection results of the photoelectric detection module and display analysis results on the display, and control the drive module and the temperature control module.

3. The device of claim 1, wherein the pre-processing module is configured to process a solvent solution to obtain the extraction solvent.

4. The device of claim 1, wherein the centrifugal system is a centrifugal detector.

5. The device of claim 1, wherein the microfluidic chip comprises a channel layer and a cover layer arranged above the channel layer;
wherein the channel layer comprises a channel layer main body and a plurality of channel branches arranged on the channel layer main body; each of the plurality of channel branches comprises a first solvent injection port, a sample inlet, an extraction well, a microchannel, a filtration well, a detection well, a waste collection well and a first air hole; an inlet of the extraction well is connected to the first solvent injection port and the sample inlet; an outlet of the extraction well is connected to an inlet of the microchannel; an outlet of the microchannel is connected to an inlet of the filtration well; an outlet of the filtration well is connected to an inlet of the detection well; an outlet of the detection well is connected to an inlet of the waste collection well; the waste collection well is communicated with the first air hole; the extraction well is provided with a plurality of heating columns; the filtration well is provided with a microarray and a plurality of microbeads; the plurality of microbeads are located above the microarray; and a filter pad is provided at the outlet of the filtration well.

6. The device of claim 5, wherein the cover layer is provided with a first mounting hole, a plurality of sample injection holes, a plurality of second solvent injection ports and a plurality of second air holes; a middle of the channel layer main body is provided with a second mounting hole corresponding to the first mounting hole; the plurality of sample injection holes, the plurality of second solvent injection ports, the plurality of second air holes and the plurality of channel branches are the same in number, and the plurality of channel branches are in one-to-one correspondence with the plurality of sample injection holes, the plurality of second solvent injection ports and the plurality of second air holes; the plurality of sample injection holes are in one-to-one correspondence with sample inlets of the plurality of channel branches; the plurality of second air holes are in one-to-one correspondence with first air holes of the plurality of channel branches; and the plurality of second solvent injection ports are in one-to-one correspondence with first solvent injection ports of the plurality of channel branches.

7. The device of claim 5, wherein the cover layer is provided with an observation window; and the observation window comprises a penetration hole arranged on the cover layer and an optically-permeable film mounted in the penetration hole.

8. The device of claim 5, wherein a base plate layer is provided below the channel layer; and a middle of the base plate layer is provided with a mounting hole.

9. The device of claim 5, wherein the filter pad is configured as at least one layer; and the filter pad is a metal filter screen, a non-metal filter cloth, a filter membrane, or a combination thereof.

10. A detection method using the device of claim 1, comprising:
(S1) processing, by the pre-processing module, a soil sample to obtain a soil sample solution;
(S2) injecting the soil sample solution and an extraction solvent into the microfluidic chip;
(S3) heating the microfluidic chip using a heating plate;
(S4) starting the centrifugal system to generate a centrifugal force to drive the soil sample solution and the extraction solvent in the microfluidic chip to flow along a channel branch for mixing and extraction, so as to obtain an extract; and
(S5) detecting, by the photoelectric detection module, the extract to determine the organic matter content in the soil sample solution.

11. A centrifugal microfluidic chip, comprising:
a channel layer;
a cover layer arranged above the channel layer; and
a base plate layer arranged below the channel layer;
wherein the channel layer comprises a channel layer main body and a plurality of channel branches arranged on the channel layer main body; each of the plurality of channel branches comprises a sample inlet, an extraction well, a microchannel, a filtration well, a detection well, and a waste collection well; an inlet of the extraction well is connected to the sample inlet; an outlet of the extraction well is connected to an inlet of the microchannel; an outlet of the microchannel is connected to an inlet of the filtration well; an outlet of the filtration well is connected to an inlet of the detection well; and an outlet of the detection well is connected to an inlet of the waste collection well.

12. The centrifugal microfluidic chip of claim 11, wherein the extraction well is provided with a plurality of heating columns.

13. The centrifugal microfluidic chip of claim 11, wherein the filtration well is provided with a microarray and a plurality of microbeads; the plurality of microbeads are located above the microarray; and a filter pad is provided at the outlet of the filtration well.

14. The centrifugal microfluidic chip of claim 11, wherein the cover layer is provided with a first mounting hole, a plurality of sample injection holes, a plurality of first solvent injection ports, and a plurality of first air holes.

15. The centrifugal microfluidic chip of claim 14, wherein each of the plurality of channel branches further comprises a second air hole connected to the waste collection well and a second solvent injection port connected to the inlet of the extraction well; a middle of the channel layer main body is provided with a second mounting hole corresponding to the first mounting hole; the plurality of sample injection holes, the plurality of first solvent injection ports, the plurality of first air holes, and the plurality of channel branches are the same in number, and the plurality of channel branches are in one-to-one correspondence with the plurality of sample injection holes, the plurality of first solvent injection ports and the plurality of first air holes; the plurality of sample injection holes are in one-to-one correspondence with sample inlets of the plurality of channel branches; the plurality of first air holes are in one-to-one correspondence with second air holes of the plurality of channel branches; and the plurality of first solvent injection ports are in one-to-one correspondence with second solvent injection ports of the plurality of channel branches.

16. The centrifugal microfluidic chip of claim 11, wherein the cover layer is provided with an observation window; and the observation window comprises a penetration hole arranged on the cover layer and an optically-permeable film mounted in the penetration hole.

17. The centrifugal microfluidic chip of claim 11, wherein a middle of the base plate layer is provided with a mounting hole.

18. The centrifugal microfluidic chip of claim 11, wherein the channel layer comprises a first channel layer and a second channel layer arranged in sequence; an upper portion of each of the plurality of channel branches is located in the first channel layer, and a lower portion of each of the plurality of channel branches is located in the second channel layer; and the upper portion is through in the first channel layer and is communicated with the lower portion in the second channel layer.

19. The centrifugal microfluidic chip of claim 13, wherein the filter pad is configured as at least one layer; and the filter pad is a metal filter screen, a non-metal filter cloth, a filter membrane, or a combination thereof.

20. A method for preparing the centrifugal microfluidic chip of claim 11, comprising:
(a) drawing patterns of microstructures required on the cover layer, the channel layer and the base plate layer by using a computer software;
(b) forming the microstructures on the cover layer, the channel layer and the base plate layer by microfabrication; and
(c) subjecting the cover layer, the channel layer and the base plate layer to alignment, bonding, and pressurized sealing to fabricate the centrifugal microfluidic chip.
